(19) United States
(12) Patent Application Publication
(10) Pub. No.: US 2026/0041940 A1
(43) Pub. Date: Feb. 12, 2026

(54) Device for Non-Invasive Ultrasonic Treatment of a Target Region Within a Body Part of a User

(71) Applicant: Fraunhofer-Gesellschaft zur Förderung der angewandten Forschung e.V., Munich (DE)

(72) Inventors: Steffen TRETBAR, Sulzbach (DE); Christian DEGEL, Sulzbach (DE)

(21) Appl. No.: 19/291,849

(22) Filed: Aug. 6, 2025

(30) Foreign Application Priority Data
Aug. 9, 2024 (DE) 102024122859.8

(51) Int. Cl. A61N 7/00; A61B 5/055

(52) U.S. Cl. CPC A61N 7/00; A61B 5/055; A61N 2007/0026; A61N 2007/006; A61N 2007/0078

(57) ABSTRACT

A apparatus for non-invasive ultrasonic treatment of a target region within a body part of a user is proposed. The apparatus includes an attachment means for attaching the apparatus to the body part. Further, the apparatus includes at least one ultrasonic transducer configured to emit ultrasonic waves for non-invasive ultrasonic treatment of the target region. For compensating for distortions of the emitted ultrasonic waves by anatomical structures of the body part and for focusing the ultrasonic waves into the target region, the apparatus comprises a modification means individually adapted to the anatomical structures of the body part of the user or alternatively a plurality of ultrasonic transducers and a control circuit. The modification means is configured to modify acoustic properties of the emitted ultrasonic waves in a predetermined manner. The control circuit is configured to control the emission of the ultrasonic waves by the plurality of ultrasonic transducers based on data indicating the target region and properties of the anatomical structures of the body part of the user. The apparatus is configured for operation in the magnetic field of a magnetic resonance imaging system.

### CROSS-REFERENCE TO RELATED APPLICATION

This application claims the benefit of priority under 35 U.S.C. § 119 to German Patent Application No. 10 2024 122 859.8, filed on Aug. 8, 2024, the entire contents of which are incorporated herein by reference.

### TECHNICAL FIELD

The present disclosure relates to non-invasive ultrasonic treatment. In particular, examples of the present disclosure relate to an apparatus for non-invasive ultrasonic treatment of a target region within a body part of a user and to a system comprising the apparatus.

### BACKGROUND

Neurological and psychiatric diseases are often characterized by a disturbed balance of the cerebral excitability of various areas of the brain. The therapeutic principle of positively influencing the disturbed balance by targeted stimulation or inhibition of areas of the brain is derived therefrom. For this purpose, electrical cerebral deep brain stimulation is currently used successfully in different neurological clinical pictures (e.g. Parkinson's disease or dystonia). It is based on the use of electrodes which are introduced directly into dysfunctional areas of the brain in order to exert a regulating influence on the signal paths there by means of electrical pulses. However, this method is invasive. Serious complications such as bleeding or infections can occur as a result of the penetration of the brain tissue. As a result of this, this invasive and risky approach is not suitable for many diseases or symptoms of diseases.

Non-invasive approaches for neuromodulation have been developed in recent years. These are, in particular, transcranial magnetic stimulation (TMS), transcranial direct current stimulation (tDCS) and optogenetics. However, these methods have serious disadvantages, such as poor spatial specificity, limitation to structures of the brain close to the surface or, as in the case of optogenetics, are also invasive.

The treatment of other regions of the body is afflicted with similar problems.

Against this background, it is an object to enable improved non-invasive treatment of the human body.

### SUMMARY

The object is achieved according by an apparatus for non-invasive ultrasonic treatment of a target region within a body part of a user and a system having the apparatus according to the independent claims. Further aspects and developments are described in the dependent claims, the following description and in the figures.

According to a first aspect, the present disclosure relates to an apparatus for non-invasive ultrasonic treatment of a target region within a body part of a user. The apparatus comprises an attachment means for attaching the apparatus to the body part. Further, the apparatus comprises at least one ultrasonic transducer configured to emit ultrasonic waves for non-invasive ultrasonic treatment of the target region. For compensating for distortions of the emitted ultrasonic waves by anatomical structures of the body part and for focusing the ultrasonic waves into the target region, the apparatus comprises a modification means individually adapted to the anatomical structures of the body part of the user or alternatively a plurality of ultrasonic transducers and a control circuit. The modification means is configured to modify acoustic properties of the emitted ultrasonic waves in a predetermined manner. The control circuit is configured to control the emission of the ultrasonic waves by the plurality of ultrasonic transducers based on data indicating the target region and properties of the anatomical structures of the body part of the user.

According to a second aspect, the present disclosure relates to a system comprising an apparatus for non-invasive ultrasonic treatment of a target region within a body part of a user according to the first aspect and a position tracking system. The position tracking system is configured to determine a position of the apparatus relative to a target position on the body part of the user and to output information about the position of the apparatus relative to the target position.

In contrast to known invasive treatment approaches, the proposed apparatus enables non-invasive treatment. This considerably reduces the risk of complications and makes the method accessible to a wider user group. By using individually adapted modification means or a plurality of ultrasonic transducers with precise control, the apparatus can accurately focus the ultrasonic waves onto the target region in a body part such as the head. This enables targeted treatment of specific regions within the body region such as targeted stimulation or inhibition of specific areas of the brain in order to correct the disturbed balance of the cerebral excitability. The apparatus is able to compensate for distortions by anatomical structures of the body part. This means that the treatment can be performed effectively and precisely despite individual anatomical differences. In comparison to other non-invasive methods such as TMS or tDCS, which often have only limited spatial specificity and are limited to structures close to the surface, the ultrasonic treatment offers better penetration depth and spatial resolution. This enables deeper and more specific influencing of the brain structures. This applies analogously to other body parts. By the possibility of individually controlling the emission of the ultrasonic waves based on data about the target region and the anatomical structures, the treatment can be flexibly adapted to the specific needs and anatomical conditions of the user. In summary, the proposed apparatus enables targeted, deep and specific treatment of the human body without the risks of invasive intervention.

### BRIEF DESCRIPTION OF THE FIGURES

Some examples of devices and/or methods are explained in more detail below merely by way of example with reference to the accompanying figures. In the figures:

FIG. 1 schematically shows a first example of an apparatus for non-invasive ultrasonic treatment;

FIG. 2 schematically shows a second example of an apparatus for non-invasive ultrasonic treatment;

FIG. 3 schematically shows an exemplary communication of an apparatus for non-invasive ultrasonic treatment with an external entity;

FIG. 4 schematically shows a third example of an apparatus for non-invasive ultrasonic treatment;

FIG. 5 schematically shows a fourth example of an apparatus for non-invasive ultrasonic treatment;

FIG. 6 schematically shows a non-invasive ultrasonic treatment with multiple apparatuses for non-invasive ultrasonic treatment;

FIG. 7 schematically shows an example of a system comprising an apparatus for non-invasive ultrasonic treatment and a position tracking system; and

FIG. 8 schematically shows a fifth example of an apparatus for non-invasive ultrasonic treatment.

### DESCRIPTION

Some examples will now be described in more detail with reference to the accompanying figures. However, further possible examples are not limited to the features of these detailed embodiments. These may include modifications to the features and equivalents and alternatives to the features. Furthermore, the terminology used herein to describe particular examples is not intended to be limiting of further possible examples.

Like or similar reference numbers refer throughout the description of the figures to like or similar elements or features, which may each be implemented identically or in modified form while providing the same or a similar function. In the figures, the thicknesses of lines, layers and/or regions may further be exaggerated for clarity.

When two elements A and B are combined using an “or,” it is to be understood that all possible combinations are disclosed, i.e., only A, only B, and A and B, unless specifically defined otherwise in the individual case. As an alternative wording for the same combinations, “at least one of A and B” or “A and/or B” may be used. This applies equivalently to combinations of more than two elements.

When a singular form, e.g., “a,” “an” and “the” is used and the use of only a single element is neither explicitly nor implicitly defined as mandatory, further examples may also use a plurality of elements to implement the same function. When a function is described below as being implemented using a plurality of elements, further examples may implement the same function using a single element or a single processing entity. It will be further understood that the terms “comprises,” “comprising,” “includes” and/or “including,” when used, describe the presence of stated features, integers, steps, operations, processes, elements, components and/or a group thereof, but do not preclude the presence or addition of one or more other features, integers, steps, operations, processes, elements, components and/or a group thereof.

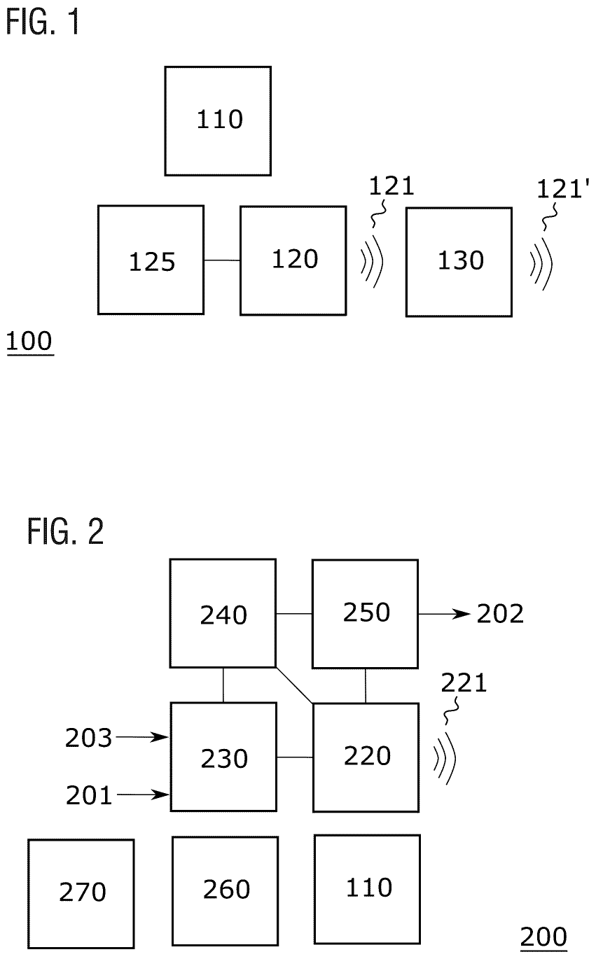

FIG. 1 schematically shows an apparatus 100 for non-invasive ultrasonic treatment of a target region within a body part of a user.

The body part may be any delineated portion of the human body that performs a particular function or represents a particular anatomical structure. For example, the body part may be the head, trunk or limb such as an arm, hand, leg or foot.

The target region is a specific region within the body part intended for non-invasive ultrasonic treatment. This target region may vary depending on the application and may be, for example, a specific region in the brain or in another organ or tissue of the body to be treated by the ultrasonic waves. In other words: the target region is the specific region in the body of the user to be influenced by the ultrasonic treatment in order to achieve, for example, therapeutic effects or effects on the well-being of the user.

The term “non-invasive” means in the context of the present disclosure that an action or treatment is performed without penetration into the body by cuts, incisions or other methods penetrating the skin or body openings.

The term “ultrasonic treatment” describes the medical or therapeutic application of ultrasonic waves or an ultrasonic signal to treat a condition of the user (e.g. a health condition or a sensation condition). For example, the ultrasonic treatment may be stimulation of the target region such as neurostimulation of specific regions of the brain of the user (e.g. for clinical pictures such as depression, addictive disorders, Parkinson's disease, dystonia, Alzheimer's disease or epilepsy), neurostimulation of peripheral nerves of the user, neurostimulation for wellness purposes (e.g. for assisting a meditation) by means of ultrasonic. Alternatively, the ultrasonic treatment may be, for example, ablation or opening of the blood-brain barrier by means of ultrasonic. However, the proposed technology is not limited to the aforementioned examples. In principle, the apparatus 100 may be used for any ultrasonic treatment of a target region in any body part of the user.

The apparatus 100 comprises an attachment means (attachment apparatus, attachment structure) 110 for attaching the apparatus 100 to the body part. The attachment means 110 is an apparatus or a mechanism configured to attach the apparatus 100 to the body part. In particular, the attachment means 110 may be configured to hold the apparatus in a fixed position on the body part in order to ensure a precise application and effect of the ultrasonic treatment. The attachment means 110 may be embodied in a variety of ways. The structure may depend, for example, on the specific ultrasonic treatment and/or on properties of the body part to which the attachment means 110 is to attach the apparatus 100. Since the attachment means 110 may be embodied in a variety of ways, it is only illustrated symbolically by the box 110 in FIG. 1.

For example, the attachment means 110 may comprise adjustable straps or straps which can be placed around the body part of the user and tightened in order to hold the apparatus 100 (e.g. stationary) on the body part. Alternatively or additionally, the attachment means 110 may comprise mechanical clamps or clips in order to attach the apparatus 110 directly to the skin or to items of clothing. Furthermore, alternatively or additionally, the attachment means 110 may comprise (e.g. flexible) tapes with hook-and-loop fasteners which are easy to attach and remove and offer an adjustable fixation. Likewise, the attachment means 110 may alternatively or additionally comprise self-adhesive pads or plasters which can be applied to the skin and fix the apparatus 100 thereto. The attachment means 110 may furthermore alternatively or additionally comprise a (e.g. flexible or rigid) cap which can be placed over the head of the user in order to hold the apparatus 100 at a predetermined position on the head of the user. Likewise, the attachment means 110 may alternatively or additionally comprise a helmet in order to hold the apparatus 100 at a predetermined position on the head of the user.

As can be seen from the aforementioned examples, according to examples the attachment means 110 may be configured for non-invasively attaching the apparatus 100 to the body part of the user. The attachment means 110 may be individually adapted to the body part of the user in order to enable a unique positioning of the apparatus 100 on the body part.

The apparatus 100 furthermore comprises at least one ultrasonic transducer 120 (i.e. N≥1 ultrasonic transducer). The at least one ultrasonic transducer 120 is configured to selectively convert electrical energy into ultrasonic waves or an ultrasonic signal 121 and then emit them for non-invasive ultrasonic treatment of the target region. The ultrasonic waves or the ultrasonic signal 121 may have e.g. a frequency of 20 kHz or more and 1 GHz or less. For example, the at least one ultrasonic transducer 120 may generate the ultrasonic waves or the ultrasonic signal 121 utilizing the piezoelectric effect or electromagnetic mechanisms. The at least one ultrasonic transducer 120 may be e.g. a micromechanical ultrasonic transducer such as a piezoelectric micromechanical ultrasonic transducer (PMUT) or a capacitive micromechanical ultrasonic transducer (CMUT). The at least one ultrasonic transducer 120 may be manufactured from different materials such as semiconductor material, plastics or combinations thereof. In particular, the at least one ultrasonic transducer 120 may be a single single-element ultrasonic transducer.

For controlling the at least one ultrasonic transducer 120, the apparatus 100 may comprise for example a control circuit 125 (which may also be interpreted as a transmission circuit) which is coupled to the at least one ultrasonic transducer 120. For example, the control circuit 125 may be controlled by a processor, a computer processor (CPU=central processing unit), a microcontroller, an application-specific integrated circuit (ASIC=application-specific integrated circuit), an integrated circuit (IC=integrated circuit), a single-chip system (SoC=system on a chip), a programmable logic element or a field-programmable gate array (FPGA) having a microprocessor on which software for controlling one or more components of the apparatus 100 runs according to the principles described herein. Furthermore, the control circuit 125 may include or be coupled to one or more memories.

The control circuit 125 is configured to generate a respective control signal for the at least one ultrasonic transducer 120. The control circuit 125 is configured to generate the electrical transmission patterns and pulses for driving the at least one ultrasonic transducer 120 and to bring the respective control signal to a corresponding voltage level. This may comprise at least one of phase, frequency, amplitude and pulse width modulations. The respective control signal for the at least one ultrasonic transducer 120 correspondingly indicates the electrical transmission patterns and pulses for driving the at least one ultrasonic transducer 120. In other words: the control circuit 125 predetermines the transmission pattern for the respective ultrasonic signal emitted by the at least one ultrasonic transducer 120. In this case, the transmission pattern indicates the time profile or the modulation of the respective ultrasonic waves emitted by the at least one ultrasonic transducer 120.

(Signal) characteristics of the respective ultrasonic waves emitted by the at least one ultrasonic transducer 120 (or of the ultrasonic signal) 121 such as amplitude or modulation may be predetermined. For example, the predetermined characteristics of the respective ultrasonic waves 121 emitted by the at least one ultrasonic transducer 120 may be programmed or stored in a memory of the apparatus 100 and the control circuit 125 may be configured to drive the ultrasonic transducer 120 according to the programmed characteristics. Correspondingly, it may be ensured that the at least one ultrasonic transducer 120 emits ultrasonic waves 121 with the predetermined characteristics for non-invasive ultrasonic treatment of the target region.

According to examples, the control circuit 125 may also be integrated into the at least one ultrasonic transducer.

For compensating for distortions of the emitted ultrasonic waves 121 by anatomical structures of the body part and for focusing the ultrasonic waves 121 into the target region, the apparatus 100 comprises a modification means (modification apparatus, modification structure) 130 individually adapted to the anatomical structures of the body part of the user, which modification means is configured to modify or predistort acoustic properties of the emitted ultrasonic waves in a predetermined manner. The ultrasonic waves after the modification or predistortion by the modification means 130 are characterized by the reference symbol 121′ in FIG. 1.

The term “anatomical structures of the body part” is understood to mean the tissues and physical features that together make up the body part. For example, this may comprise, for the respective body part, one or more of bones, muscles, skin, adipose tissue, blood vessels, nerves, organs, cartilage and connective tissue. These distort the emitted ultrasonic waves 121 via different mechanisms. Some examples are mentioned below. However, the collection of examples is not exhaustive. Different tissue types have different sound velocities and densities. When ultrasonic waves transition from one tissue to another (e.g. transition from soft tissue such as skin and fat to denser tissue such as bones), refractions and reflections may occur. Ultrasonic waves may be reflected or scattered at interfaces between different tissue types (e.g. reflection at the surface of bones or scattering by heterogeneous tissue such as muscles). Air bubbles or air-filled cavities (e.g. lung or gastrointestinal tract) also reflect ultrasonic waves. This may lead to a distribution of the energy and impair the focusing of the waves. Different tissues absorb ultrasonic waves to different degrees (e.g. absorption of ultrasonic waves by muscular or fatty tissue). A higher absorption may reduce the energy of the waves before they reach the target region. The shape and structure of anatomical features may influence the propagation direction and focusing of the ultrasonic waves (e.g. curvature and unevenness of bone structures or other organic surfaces). Fluids in the body (e.g. blood vessels or cysts) sometimes have a different sound velocity and may redirect, de-focus or re-focus the ultrasonic waves. Distortions of the emitted ultrasonic waves 121 by anatomical structures of the body part may impair the efficacy of the treatment overall.

This may be compensated for by the modification means 130. As a result of the modification means 130 being individually adapted to the anatomical structures of the body part of the user, the ultrasonic waves 121 may be modified or predistorted in such a manner that the distortions of the emitted ultrasonic waves 121 by anatomical structures of the body part are at least partially or (substantially) completely compensated for. For this purpose, the modification means 130 is configured to adapt one or more characteristics of the emitted ultrasonic waves 121 such as shape, direction or intensity in order to correct distortions by the anatomical structures of the body part of the user. Further, the modification means 130 is configured to focus one or more characteristics of the emitted ultrasonic waves 121 into the target region by modification thereof.

For example, the modification means 130 may comprise at least one of an ultrasonic lens and an acoustic phase plate for predetermined modification of the acoustic properties of the emitted ultrasonic waves 121. In other words: the modification means 130 may comprise at least one ultrasonic lens and/or at least one acoustic phase plate.

Similarly to an optical lens influencing light waves, an ultrasonic lens acts on ultrasonic waves in order to achieve precise focusing and control of the ultrasonic waves. The ultrasonic lens is configured to collect the ultrasonic waves 121 and to concentrate them onto a specific point or region. The ultrasonic lens thus enables the focusing of the ultrasonic waves into the target region. By changing the shape and propagation direction of the emitted ultrasonic waves 121, the ultrasonic lens may also help to correct distortions caused by different tissue types or other anatomical structures. The ultrasonic lens is manufactured from one or more materials which enable good sound transmission and at the same time effect the desired modification of the ultrasonic waves 121. For example, the ultrasonic lens may be manufactured from plastics and/or ceramics which have acoustic properties suitable for the focusing of ultrasonic.

Acoustic phase plates are devices which modify the phase of ultrasonic waves by the manipulation of the propagation speed and the wave fronts of the ultrasonic waves. This enables precise control of the emitted ultrasonic waves 121. The acoustic phase plate is configured to change the phase of the ultrasonic waves passing through it. This means that the phase position of the wave fronts is shifted relative to one another. By adapting the phase position of the ultrasonic waves, the acoustic phase plate may correct distortions caused by the anatomical structures of the body part of the user. By modifying the phase, the ultrasonic waves may also be adapted to be focused in a specific region. The acoustic phase plate thus enables the focusing of the ultrasonic waves 121 into the target region. The acoustic phase plate may be manufactured from different materials which have specific acoustic properties in order to achieve the desired phase modification of the emitted ultrasonic waves 121. For example, the acoustic phase plate may be manufactured from a metal such as titanium or aluminum, a plastic such as polymethyl methacrylate (PMMA) or polyether ether ketone (PEEK), a ceramic such as lead zirconate titanate (PZT) or aluminum oxide (alumina), a composite such as fiber-reinforced plastics, quartz, a polymer such as poly vinyl den fluoride (PVDF) or a combination of one or more thereof. The design or geometric dimensions (e.g. thickness or shape) of the acoustic phase plate are determined by how the acoustic phase plate is to change the phase of the ultrasonic waves 121.

The apparatus 100 enables non-invasive ultrasonic treatment. In comparison to known invasive treatment approaches, this considerably reduces the risk of complications and makes ultrasonic treatment accessible to a wider user group. By using the individually adapted modification means 130, the apparatus 100 can accurately focus the ultrasonic waves 121 onto the target region in a body part such as the head. This enables targeted treatment of specific regions within the body region such as targeted stimulation or inhibition of specific areas of the brain in order to correct the disturbed balance of the cerebral excitability. The apparatus 100 is able to compensate for distortions by anatomical structures of the body part. This means that the ultrasonic treatment can be performed effectively and precisely despite individual anatomical differences. In comparison to other non-invasive methods such as TMS or tDCS, which often have only limited spatial specificity and are limited to structures close to the surface, the ultrasonic treatment offers better penetration depth and spatial resolution. This enables deeper and more specific influencing of the brain structures by means of the apparatus 100. This applies analogously to other body parts. Thus, the apparatus 100 enables targeted, deep and specific ultrasonic treatment of the human body without the risks of invasive intervention.

According to examples, the modification means 130 may be replaceably arranged. In other words: the modification means 130 and the apparatus 100 are designed such that the modification means 130 can be (simply) removed, re-inserted or replaced by another modification means. The possibility of exchanging the modification means 130 increases the flexibility of the apparatus 100. The user can adapt the apparatus 100 to different treatment scenarios by using different modification means. The possibility of easily exchanging the modification means 130 simplifies the maintenance of the apparatus 100. Should the modification means 130 become worn or damaged, it can be quickly and easily replaced. Due to the exchangeability of the modification means 130, the service life of the apparatus 100 can be extended, since only the modification means 130 and not the entire apparatus 100 has to be replaced. This reduces the long-term costs for the user. If the anatomical structures of the body part of the user change (e.g. due to an increase or decrease in weight or a surgical intervention), this circumstance can be easily countered by exchanging the modification means 130 by a modification means adapted to the changed anatomical structures. Due to the exchangeability of the modification means 130, the apparatus 100 can also be used more flexibly for multiple users. Each user can use his own, adapted modification means. This makes the apparatus 100 particularly useful in community or clinical environments, where it can be used for different persons, without compromising the adaptation and effectiveness of the ultrasonic treatment.

According to an exemplary embodiment, the ultrasonic transducer 120 may consist of only one single-element ultrasonic transducer and one electronic (transmission) channel. In order to compensate for the user-specific anatomical circumstances (e.g. shape of the skull bone/calotte or acoustic properties), the apparatus in this exemplary embodiment is then designed in such a manner that specific user-specific compensation means 130 (e.g. phase plates, lenses) which adapt the sound field by their acoustic properties are integrated in front of the ultrasonic transducer 120 in a fixed but possibly exchangeable manner (e.g. for other anatomical circumstances or patients).

An alternative for compensating for distortions of the emitted ultrasonic waves by anatomical structures of the body part and for focusing the ultrasonic waves into the target region is shown in FIG. 2. Substantially the differences between the apparatus 200 shown in FIG. 2 and the apparatus 100 shown in FIG. 1 for non-invasive ultrasonic treatment of a target region within a body part of a user are described below.

The apparatus 200 dispenses with the modification means 130. In other words: the apparatus 200, unlike the apparatus 100, does not comprise the modification means 130.

While the apparatus 100 comprises at least one ultrasonic transducer, the apparatus 200 comprises a plurality of ultrasonic transducers (i.e. M>2 ultrasonic transducers) 220. The individual ultrasonic transducers of the plurality of ultrasonic transducers 220 may in this case be embodied as described above for the at least one ultrasonic transducer 120. The ultrasonic transducers of the plurality of ultrasonic transducers 220 may in this case be arranged substantially as desired, e.g. as a ring array (annular ring array), as a 1 D array, as a 1.5 D array, as a 1.75 D array or as a 2 D array (e.g. a matrix array).

The control circuit 125 is replaced by the control circuit 230 which is coupled to the plurality of ultrasonic transducers 220. The control circuit 230 may be implemented analogously to the control circuit 125. The control circuit 230 is configured to control the emission of the ultrasonic waves 221 by the plurality of ultrasonic transducers 220 based on data 201 indicating (displaying, representing or encoded with) the target region and properties of the anatomical structures of the body part of the user. With regard to the target region, the data 201 may, for example, indicate the (exact) coordinates or location of the target region in the body of the user. Likewise, the data 201 may indicate the size (dimensions) and the (e.g. three-dimensional) shape of the target region. This information enables precise alignment and focusing of the ultrasonic wave 221 into the target region. With regard to the properties of the anatomical structures of the body part of the user, the data 201 may, for example, indicate the type of tissue (e.g. muscle, fat, tumor, bone) and furthermore location, size and shape of the respective tissue in the body region lying between the apparatus 200 and the target region and optionally furthermore in the target region. Alternatively or additionally, the data 201 may indicate (e.g. measured or simulated) acoustic properties such as (frequency-dependent) reflectivity, (frequency-dependent) attenuation, (frequency-dependent) sound velocity of the tissue in the body region lying between the apparatus 200 and the target region and optionally furthermore in the target region.

The ultrasonic waves emitted by the individual ultrasonic transducers of the plurality of ultrasonic transducers 220 are superimposed so that the resulting superimposed ultrasonic waves 221 may be adjusted by adjusting the characteristics of the ultrasonic waves emitted by the individual ultrasonic transducer with regard to their characteristics for compensating for distortions by anatomical structures of the body part and for focusing into the target region. The target region and properties of the anatomical structures of the body part of the user are known from the data 201 so that the control circuit 230 may adjust the characteristics of the ultrasonic waves emitted by the individual ultrasonic transducers of the plurality of ultrasonic transducers 220 correspondingly (e.g. according to a mechanistic, i.e. rule-based model, a trained machine learning model or a combination thereof). The control circuit 230 may, for example, be configured to set, based on the data 201, at least one of amplitude, phase, frequency and transmission pattern of the ultrasonic waves individually for the respective ultrasonic transducer of the plurality of ultrasonic transducers 220. By precisely adapting the phases, the emitted ultrasonic waves 221 may be controlled to meet at a specific point or region in the body and constructively interfere there to generate focused ultrasonic energy. In addition to the phase control, the control circuit may adapt the amplitude and frequency of the ultrasonic waves in order to control the intensity and penetration depth of the waves. The control circuit 230 may generate different transmission patterns in order to follow different treatment strategies. For example, it may use continuous or pulsed ultrasonic waves depending on which type of treatment is required. This enables dynamic and precise adaptation of the ultrasonic waves 221 for optimal ultrasonic treatment.

In other words, the control circuit 230 may enable, inter alia, the variation of the sound intensity, the treatment position (e.g. x-, y-, z-coordinate) and/or the treatment duration as well as any transmission sequence patterns and frequencies (e.g. for specific clinical pictures and/or user-specific situations).

By using a plurality of ultrasonic transducers and precisely controlling their emissions, the ultrasonic waves 221 can be very accurately focused onto the target region. This increases the efficacy of the treatment and minimizes undesired effects on surrounding tissue. By accurately focusing and controlling the ultrasonic waves 221, higher energy levels can be used without damaging the surrounding tissue. This improves the safety and effectiveness of the ultrasonic treatment. The control circuit 230 enables dynamic adaptation of the ultrasonic waves 221 based on real-time data about the target region and the anatomical structures. This ensures individual adaptability of the ultrasonic treatment to the respective user. The control of the phases and amplitudes of the ultrasonic waves 221 helps to compensate for distortions caused by different tissue types and/or other anatomical structures. The apparatus 200 can be used for a wide range of (e.g. medical) applications, including the treatment of deep-lying tissues or organs which are difficult to achieve with conventional methods.

In the example of FIG. 2, for highly accurate focusing of the ultrasonic into the target region, the user-specific anatomical circumstances (e.g. shape of the skull bone/calotte or acoustic properties) on the body part of the user (e.g. head) are already compensated for at the sound-emitting elements by an electronic phase-corrected transmission pattern for each individual element of the ultrasonic array. This enables the focusing of the ultrasonic in all spatial directions including the individual phase delays. For this purpose, the ultrasonic electronics provide a plurality of electronic (transmission) channels.

In some examples, the apparatus 200 may comprise only a few ultrasonic transducers (e.g. an array having a few ultrasonic transducers) and few electronic channels for cost reasons. This may be sufficient for different application cases despite limited flexibility in the ultrasonic beam shaping and control.

The data 201 may be generated by or at the apparatus 200 itself or at least partially provided by an external entity (i.e. an entity that is not part of the apparatus 200).

For example, at least one of the plurality of ultrasonic transducers 220 may be configured to emit ultrasonic waves for determining at least one property of the anatomical structures of the body part of the user. Further, at least one of the plurality of ultrasonic transducers 220 may be configured to receive reflections of the ultrasonic waves for determining at least one property of the anatomical structures of the body part of the user. The at least one of the plurality of ultrasonic transducers 220 emitting the ultrasonic waves for determining at least one property of the anatomical structures may be identical to or different from the at least one of the plurality of ultrasonic transducers 220 receiving reflections of the ultrasonic waves for determining at least one property of the anatomical structures. In other words: the apparatus 200 may be configured to calibrate one or more properties of the anatomical structures of the body part of the user itself by means of ultrasonic. For this purpose, parts or the entire plurality of ultrasonic transducers 220 (as well as the control circuit 230) may be reused. The property(s) to be determined of the anatomical structures of the body part of the user is or are one or more (e.g. all) of the properties indicated or to be indicated by the data 201 (see the aforementioned examples).

The apparatus 200 may optionally comprise a processing circuit 240 which is coupled to the control circuit 230 and the plurality of ultrasonic transducers 220. The processing circuit 240 may be implemented analogously to the control circuit 230. In some examples, the control circuit 230 and the processing circuit 240 may be formed by the same circuit. The processing circuit 240 is configured to determine the data 201 indicating the properties of the anatomical structures of the body part of the user based on the reflections measured by the at least one of the plurality of ultrasonic transducers 220. For this purpose, the processing circuit 240 is configured to receive measurement data indicating the reflections measured by the at least one of the plurality of ultrasonic transducers 220 from the at least one of the plurality of ultrasonic transducers 220 and to process these in order to determine or derive therefrom the properties of the anatomical structures of the body part of the user. For example, the processing circuit 240 may determine one or more of the properties of the anatomical structures of the body part of the user by means of a mechanistic, i.e. rule-based model, a trained machine learning model or a combination thereof, wherein the model receives the measurement data from the at least one of the plurality of ultrasonic transducers 220 as input. For example, acoustic properties such as (frequency-dependent) reflectivity, (frequency-dependent) attenuation or (frequency-dependent) sound velocity of the tissue in the body region lying between the apparatus 200 and the target region or in the target region can be determined from the measured reflections.

Alternatively or additionally, the apparatus 200 may comprise an interface 250 which is coupled at least to the processing circuit 240 and the plurality of ultrasonic transducers 220. The interface 250 may be both wired (e.g. wire-bound or fiber-optical) and wireless (wireless).

In principle, both proprietary interfaces or communication protocols and standardized interfaces or communication protocols can be used. For the wired communication, e.g. simple serial interfaces such as RS-232 can be used natively or serially via USB, the native USB interface (e.g. USB 2.0/3.X/4), special industrial bus systems, manufacturer-specific interface (e.g. USB via Lightning), LAN, CAN bus or Mil bus. For the wireless communication, e.g. WLAN, Bluetooth, simple RF radio interfaces (e.g. 433 MHz/866 MHz radio), ZigBee (Z-Wave), LoRaWAN and mobile radio standards such as 2G, 4G and 5G or future standards can be used.

The interface 250 is configured to output output data 202 indicating the reflections measured by the at least one of the plurality of ultrasonic transducers 220 to an external entity (not shown in FIG. 2). The external entity may be any system that can either further process the output data 202 itself or at least forward it to a third system for further processing. For example, the external entity may be a server, a data cloud (computing cloud), a medical diagnostic system, a computer, a laptop computer, a tablet computer or a mobile telephone. The interface 250 is further configured to subsequently (i.e. after outputting the output data 202) receive the data 201 indicating the properties of the anatomical structures of the body part of the user from the external entity. By transmitting the output data 202 to the external entity, the processing capacity can be extended. External systems may have more powerful computing resources and specialized algorithms that enable more accurate and comprehensive analyses. Conversely, the apparatus 200 does not have to be equipped with large computing resources, which can keep the manufacturing costs for the apparatus 200 low. The ability to send and receive data to and from external entities enables the integration of the apparatus into larger medical networks and diagnostic systems. This can lead to better coordination and more efficient use of resources.

According to examples, the apparatus may comprise only the processing circuit 240 and not the interface 250 or both the processing circuit 240 and the interface 250. The data 201 indicating the properties of the anatomical structures of the body part of the user can in the latter case according to examples be determined both at least partially within the apparatus 200 and at least partially outside the apparatus 200 (see the description above). In particular, a part of the data 201 can be determined by the processing circuit 240 according to the above principles and another part of the data 201 can be determined by the external entity according to the above principles.

According to examples, the data 201 indicating the properties of the anatomical structures of the body part of the user can also be received completely from the external entity, i.e. without previous measurement(s) by the apparatus 200.

The interface 250 may further be configured to receive the data 201 indicating the target region from the same or a further external entity.

For example, the data 201 indicating the target region and the properties of the anatomical structures of the body part of the user can be collected or determined on the basis of magnetic resonance tomography (MRI), computer tomography (CT) or other data sources or imaging methods.

Likewise, further data 201 indicating target characteristics for the emitted ultrasonic waves 221 (e.g. duration of the emission, transmission or sequence pattern, frequency of the ultrasonic waves) can be received from the same or a further external entity.

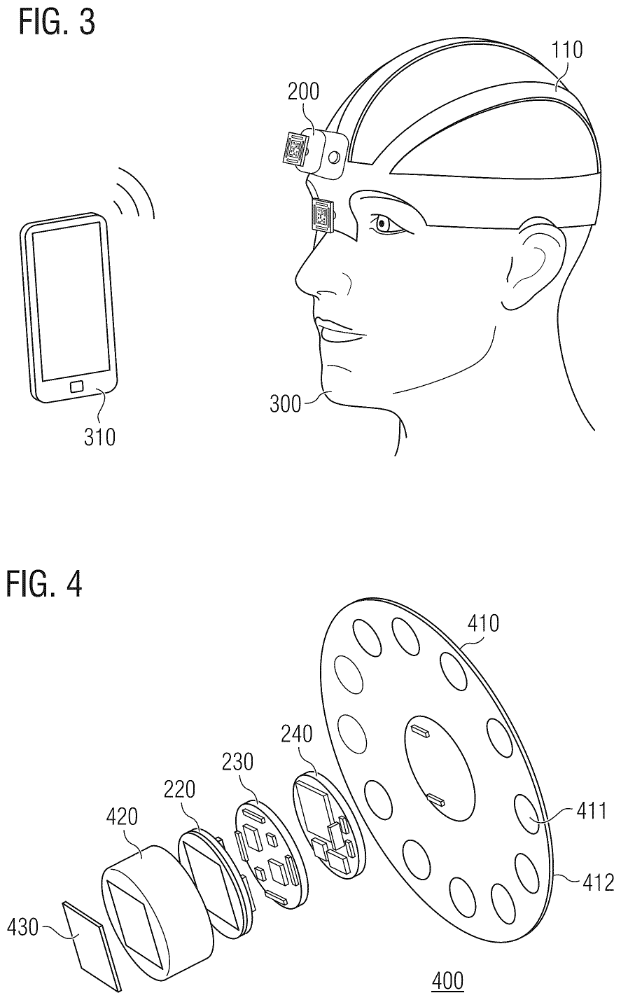

The various parameters indicated by the data 201 can be predefined e.g. partially or completely in an application on a mobile user terminal (e.g. mobile telephone or tablet computer) or computer and transmitted for example wirelessly (e.g. via WLAN, Bluetooth, etc.) via the interface 250 to the apparatus 200. This is graphically illustrated by way of example in FIG. 3, which shows the apparatus 200 in a state mounted on the head of the user together with a mobile telephone 310. The attachment means 110 is in this case embodied as a flexible cap. Via the application on the mobile telephone 310 and the interface 250 of the apparatus 200, the ultrasonic treatment can be controlled by the apparatus 200 for example in real time and one or more of the aforementioned parameters can be adapted via the data 201 during the ultrasonic treatment.

Alternatively, one or more of the aforementioned parameters can also be adjusted on the apparatus 200 itself. For this purpose, the apparatus 200 may further comprise for example a user interface 260. The user interface 260 is configured to receive a user input of the user. The user interface 260 may comprise for example mechanical input options such as buttons, pushbuttons, keys etc. in order to enable user inputs. Likewise, the user interface 260 may be designed e.g. as a touchscreen or comprise such a touchscreen in order to receive user inputs. In this case, the user interface 260 may further output information relating to the apparatus 200 or the ultrasonic treatment to the user via the touchscreen. Alternatively or additionally, the user interface 260 may comprise e.g. a microphone in order to receive user inputs in the form of speech or speech commands. The user interface 260 may, for example, be configured to receive a user input and generate the data 201 indicating the target region based on the user input. Likewise, the user interface 260 may be configured to receive one or more further or other user inputs and generate the data 201 indicating the target characteristics for the emitted ultrasonic waves 221 (e.g. duration of the emission, transmission or sequence pattern, frequency of the ultrasonic waves) based thereon.

The apparatus 100 may also optionally comprise a user interface configured analogously to the user interface 260 in order to receive one or more user inputs with target characteristics for the emitted ultrasonic waves 121 so that the control circuit 125 can drive the at least one ultrasonic transducer correspondingly.

The apparatus 200 may further be configured to adapt the ultrasonic treatment in real time on the basis of a feedback for the ultrasonic treatment. For this purpose, the control circuit 230 may further be configured to control the emission of the ultrasonic waves 221 by the plurality of ultrasonic transducers 220 based on data 203 indicating at least one physiological property of the user during the non-invasive ultrasonic treatment of the target region. The physiological property of the user is a property (size, characteristic) describing the physiology of the user. In other words: the physiological property is a property describing one or more functions and/or mechanisms in the body of the user. For example, the physiological property may be one or more of the following: a heart rate (pulse) of the user, a heart rate variability of the user, a heart cycle of the user, a respiration (respiratory rate) of the user, a blood pressure of the user, a body temperature of the user, etc. The control circuit 230 uses the physiological data 203 in order to adapt the emission of the ultrasonic waves 221 in real time. The control circuit 230 may, for example, be configured to start or end the emission of the ultrasonic waves 221 based on the data 203 and/or to emit specific ultrasonic sequences and/or to adapt an intensity of the emitted ultrasonic waves 221. By adapting the ultrasonic emissions to the current physiological properties of the user, the treatment can be tailored individually to the respective patient. This increases the effectiveness and safety of the treatment. The continuous monitoring and adaptation to physiological properties reduces the risk of side effects and undesired reactions. For example, the treatment can be automatically interrupted or adapted when a critical temperature or an unusual heart rate pattern is detected.

The apparatus 200 may itself comprise at least one sensor 270 configured to measure the at least one physiological property of the user during the non-invasive ultrasonic treatment of the target region and to generate the data 203. For example, the sensor may be an electroencephalography (EEG) sensor or a temperature sensor. Likewise, a part of the plurality of ultrasonic transducers 220 may be configured to serve as an ultrasonic sensor during the ultrasonic treatment.

FIG. 4 shows in this regard a further apparatus 400 for non-invasive ultrasonic treatment of a target region within a body part of a user. In the example of FIG. 4, the sensor is configured as an EEG sensor 410. A plurality of EEG electrodes 411 are arranged on a carrier substrate 412 of the EEG sensor 410 such that they may be attached to the body of the user during the non-invasive ultrasonic treatment of the target region and may measure an EEG of the user.

The control circuit 230 may adapt the emission of the ultrasonic waves by the plurality of ultrasonic transducers 220 according to the measured EEG of the user.

It is further shown in FIG. 4 that the plurality of ultrasonic transducers 220 and the other components of the apparatus 400 are arranged in a housing 420 of the apparatus 400 or the EEG sensor 410 is arranged on the housing 420.

FIG. 4 further shows the presence of a contact means (contact apparatus, contact structure) 430 for contacting the body part of the user. A shape of the contact means 430 is deformable to conform to a shape of the body part of the user. The contact means 430 is configured to acoustically couple the plurality of ultrasonic transducers 220 and the body part of the user. The contact means 430 is a substance or a material which facilitates the transmission of ultrasonic waves between the body part of the user and the apparatus and thus the respective ultrasonic transducer. The contact means 430 serves as a medium through which the ultrasonic waves can propagate in order to ensure that there is only the lowest possible or minimum energy loss when the ultrasonic waves pass from the respective ultrasonic transducer through the contact means 430 and then into the human being and vice versa. The contact means 430 may be e.g. an acoustic coupling medium or comprise such a medium or be filled with such a medium. The acoustic coupling medium may be e.g. a gel. Such gels are also referred to as “ultrasonic gel”, “sono gel” or “contact gel”. For example, the gel may be a water-based gel. However, the present disclosure is not limited thereto. In other examples, the acoustic coupling medium may be e.g. water, a water mixture or an oil. In general, any suitable substance may be used for the acoustic coupling medium. The composition of the acoustic coupling medium may depend, for example, on the specific application and thus on the frequency range of the emitted ultrasonic waves. Alternatively or additionally, the contact means 430 may be manufactured e.g. from flexible plastic or comprise such a plastic. The contact means 430 may be replaceably arranged.

Even if this is not explicitly illustrated in FIG. 1 and FIG. 2, each of the apparatuses 100 and 200 may also comprise a contact means configured analogously to the contact means 430, which is configured to acoustically couple the at least one ultrasonic transducer and the body part of the user.

Referring back to FIG. 2, the data 203 may alternatively or additionally be received partially or completely from the external entity described above or from another external entity. For this purpose, the interface 250 may be configured to receive the data 203 indicating the at least one physiological property of the user during the non-invasive ultrasonic treatment of the target region (at least partially) from an external entity. For example, the data 203 may be received from an external EEG sensor, an external temperature sensor, an external ultrasonic sensor or an external MRI system.

As indicated above, the treatment feedback may be a variety of ways. For example, the apparatus may comprise an EEG monitoring for treatment feedback. As illustrated in FIG. 4, for this purpose, the apparatus may have an interface for EEG electrodes 410, a holding system 412 and evaluation electronics in the form of the control and/or processing circuit 230 or 240. Likewise, an ultrasonic monitoring may be provided. This may be achieved either by means of additional ultrasonic transducers which serve as a receiving element or ultrasonic sensor or by means of the use of the already present plurality of ultrasonic transducers 220 (entirely or in parts) and of the corresponding circuit parts of the control and/or processing circuit 230 or 240. Likewise, temperature monitoring may be provided at or in the ultrasonic aperture in order to avoid possible thermal damage to the user. Integration of further monitoring systems and methods is also possible. These monitoring parameters may, as described above, be used for controlling the ultrasonic treatment in order, for example, to detect different monitoring parameters (ultrasonic, EEG signals, temperature, etc.), the reasons of which could lie, for example, in dysfunctions in the brain or another element of the body, and to allow the apparatus to react thereto (for example starting or ending the ultrasonic treatment, triggering specific ultrasonic sequences, intensity adaptation, etc.). The data processing of the monitoring parameters or signals may alternatively also be performed by the external entity, such that corresponding control commands for the apparatus can be provided via the data 201. Monitoring parameters measured at the apparatus may be transmitted to the external entity via the interface 250.

According to examples of the present disclosure, the respective apparatus (for example one of the apparatuses 100 and 200) may further comprise an accumulator configured to provide electrical energy to the at least one ultrasonic transducer (or the plurality of ultrasonic transducers) and other electrical components of the apparatus. The accumulator may be embodied in an exchangeable manner. The accumulator may, for example, be configured to be charged wirelessly (e.g. inductively). Alternatively, a charging socket may be provided on the apparatus.

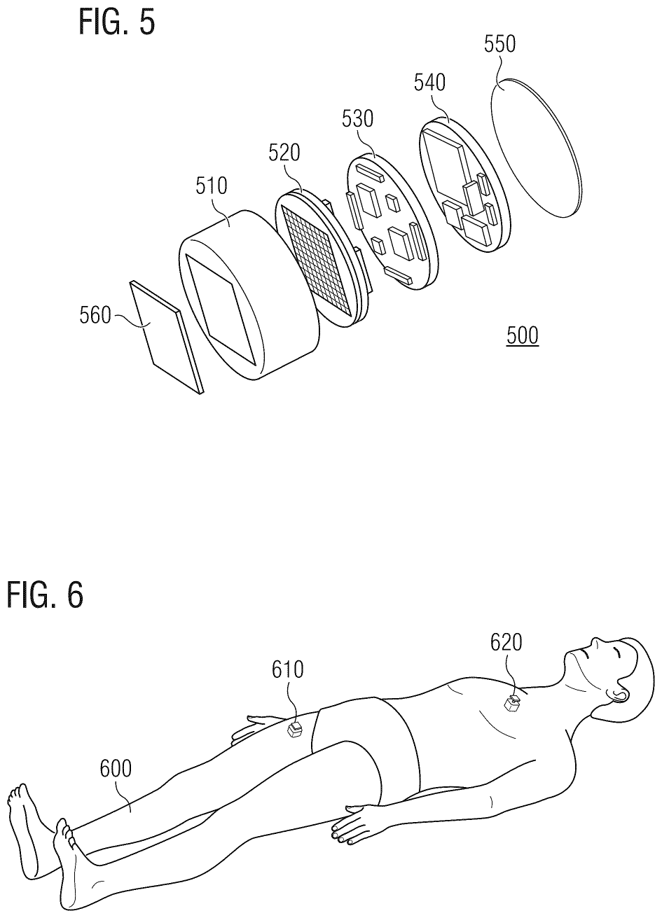

As already indicated in FIG. 4, the respective apparatus (for example one of the apparatuses 100 and 200) may comprise a housing in which the at least one ultrasonic transducer (or the plurality of ultrasonic transducers) and at least a part (for example all) of the other components of the apparatus are arranged. This is again explicitly illustrated in FIG. 5. FIG. 5 schematically shows an exploded illustration of a apparatus 500 for non-invasive ultrasonic treatment of a target region within a body part of a user.

The apparatus comprises a housing 510 and three circuit boards 520, 530 and 540 which, in the assembled state of the apparatus 500, are arranged in the housing 510. The housing 510 is closable by means of a cover 550. The at least one ultrasonic transducer or the plurality of ultrasonic transducers are arranged on the circuit board 520. The other electronics such as e.g. control circuit, processing circuit, accumulator, etc. are arranged on the circuit boards 530 and 540. The contact means 560 which is adaptable to the shape of the body part of the user is arranged in a recess 511 in the housing 510. The body part of the user is acoustically coupled to the ultrasonic transducer or transducers on the circuit board 510 via the contact means 560.

Apparatuses according to the present disclosure such as e.g. the apparatus 500 may be embodied in a compact manner. An extension of the housing 510, which extends as a three-dimensional object in three mutually perpendicular spatial directions, may for example be less than 20 cm, 15 cm, 10 cm or 5 cm in each of the three mutually perpendicular spatial directions. For example, the housing 510 may be miniaturized approximately to the size of a matching box upon integration of all the components necessary for the ultrasonic treatments.

The apparatuses described herein (e.g. one of the apparatuses 100, 200, 400 and 500) may be configured for operation in the magnetic field of an MRI system. In these examples, the materials and electronic components used for the apparatus are correspondingly selected or constructed such that they do not disturb the magnetic field of the MRI system and are themselves not negatively influenced thereby. All parts of the apparatus, in particular the attachment means and the ultrasonic transducers, may for example consist exclusively or substantially of non-magnetic materials which cause no artifacts or disturbances in the magnetic field of the MRI system. In other words: few to no ferromagnetic materials are used. Likewise, the apparatus may additionally be equipped with shields or other safety mechanisms in order to minimize interferences with the MRI system. Correspondingly, the apparatus is compatible with the MRI system and can be used therein without the apparatus and the MRI system disturbing one another.

For example, imaging of the treated body part or of the entire user may be performed during ultrasonic treatment with the MRI system. The data obtained with the MRI system can be evaluated and used to control the ultrasonic treatment. For example, the data 201 with parameters derived from the data of the MRI system (e.g. the target region) can be made available via the interface 250. Likewise, the data of the MRI system can be made available via the interface 250 and the emission of the ultrasonic waves can be correspondingly controlled by the control circuit 230 and/or the processing circuit 240 (e.g. by determining the target region from the data of the MRI system).

The apparatuses described herein (e.g. one of the apparatuses 100, 200, 400 and 500) may further comprise a camera configured to capture images of an environment of the apparatus. A processing circuit of the apparatus (e.g. the already present processing circuit 240 of the apparatus 200 or another processing circuit or additional processing circuit of the apparatus) may then be configured to determine a positioning of the apparatus relative to a target position on the body part of the user from the images of the camera and to determine instructions for assisting a positioning of the apparatus at the target position based on the determined positioning of the apparatus relative to the target position. For this purpose, the processing circuit may evaluate for example the images of the camera with a mechanistic, i.e. rule-based model, a trained machine learning model or a combination thereof. The target position is a specific point or region on the skin on the body part of the user at which the apparatus for non-invasive ultrasonic treatment is to be positioned in order to carry out the ultrasonic treatment. The target position ensures that the emitted ultrasonic waves are accurately focused onto the target region to be treated. In these examples, the apparatus may further comprise an output means (output apparatus) configured to output the instructions. The output means may, for example, be a loudspeaker, a haptic output (e.g. for vibrations) or a graphical display. This ensures that the apparatus is positioned accurately at the intended target position on the body part of the user. The automated instructions for positioning the apparatus help to minimize human errors. This leads to a more consistent and reliable placement of the apparatus, which increases the efficacy of the ultrasonic treatment.

The apparatuses described herein (e.g. one of the apparatuses 100, 200, 400 and 500) may be adapted analogously for the community operation with further sensors, e.g. an EEG sensor as described above, and may, for example, be optimized with regard to electromagnetic compatibility (EMC).

Multiple of the apparatuses described herein for non-invasive ultrasonic treatment may also be used together for ultrasonic treatment. This is shown by way of example in FIG. 6. A first apparatus 610 for non-invasive ultrasonic treatment according to the principles described herein (e.g. one of the apparatuses 100, 200, 400 and 500) is placed on the right thigh of the user 600 in order to treat a target region in the right thigh with ultrasonic. In addition, a further, second apparatus 620 for non-invasive ultrasonic treatment according to the principles described herein (e.g. one of the apparatuses 100, 200, 400 and 500) is placed on the chest of the user 600 in order to treat a target region in the upper body with ultrasonic.

The ultrasonic treatments of the target regions in the right thigh and in the upper body may also be performed in a coordinated manner by the apparatuses 610 and 620. For this purpose, the apparatuses 610 and 620 may include an interface such as the interface 250 described above in order to exchange data for the coordinated non-invasive ultrasonic treatment with one another (e.g. data about the respective target region, respective characteristics of the emitted ultrasonic waves, times or time windows of the ultrasonic emission, etc.). By the possibility of exchanging data with another apparatus, more complex treatment protocols can be performed. This is particularly useful for large-area treatments or for the treatment of multiple target regions at the same time. The coordinated control of multiple apparatuses increases the precision of the ultrasonic treatment. By synchronizing the ultrasonic waves, they can be selectively amplified or specific regions treated at the same time, which increases the effectiveness of the ultrasonic treatment. The coordinated use of multiple apparatuses can reduce the overall treatment time since parallel or sequential treatments can be optimized. This leads to more efficient use of the resources and faster patient care.

It can further be seen from FIG. 6, in particular in conjunction with FIG. 3, that apparatuses for non-invasive ultrasonic treatment according to the principles described herein can be used for ultrasonic treatment of any body parts of a user.

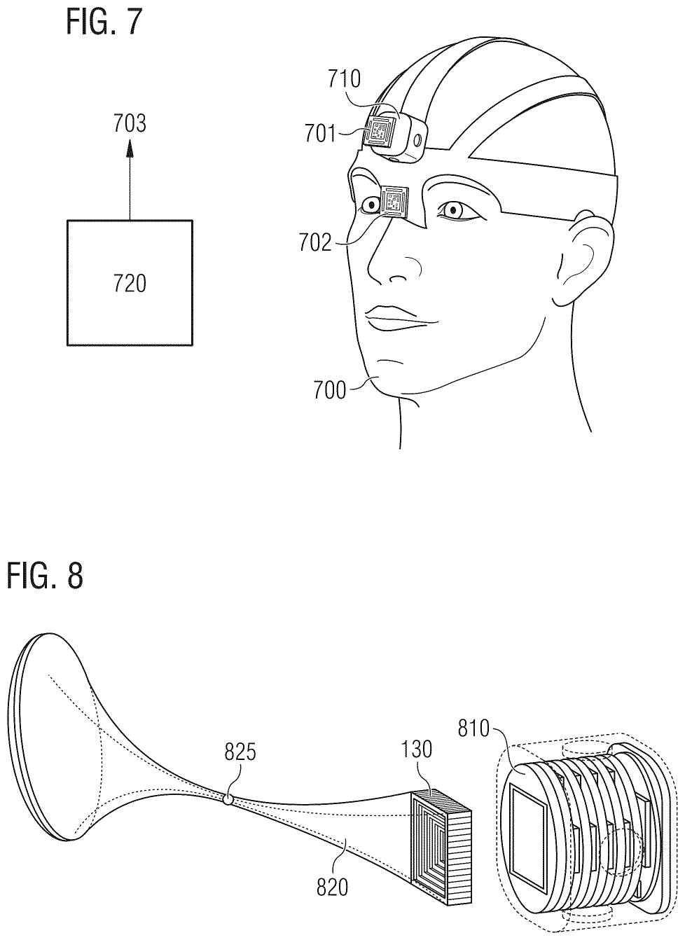

For the positioning of the apparatuses described herein for non-invasive ultrasonic treatment, an external position tracking system can also be used. A corresponding system comprising a apparatus 710 for non-invasive ultrasonic treatment according to the principles described herein (e.g. one of the apparatuses 100, 200, 400 and 500) and an external position tracking system 720 is shown in FIG. 7.

The position tracking system 720 is configured to determine a position of the apparatus 710 relative to the target position on the body part of the user 700. In the example of FIG. 7, the body part is the head of the user 700 and the target position is located on the forehead of the user 700.

A QR code 701 which is detected by the position tracking system 720 is applied to the apparatus 710. A further QR code 702 in the region between the eyes 702 of the user 700 is also illustrated by way of example as a reference in FIG. 7. Optionally, further QR codes can be used as references at further defined body positions or body sites of the user 700 (e.g. on the ears, on the chin or on the forehead in the example of FIG. 7). According to known methods, the position of the apparatus 710 relative to the target position on the body part of the user 700 can be determined via the optical detection of the QR codes 701, 702, etc.

The position tracking system 720 can also use other optical markers or also other technology for position determination instead of QR codes. In particular, known optical, inductive or acoustic methods can be used for position determination or position tracking by the position tracking system 720.

The position tracking system 720 is further configured to output information 703 about the position of the apparatus relative to the target position. This information can be output e.g. in real time in order to assist the user or medical personnel in the positioning. This can be done, for example, via a loudspeaker, a haptic output (e.g. vibrations) or a graphical display of the position tracking system 720. This ensures that the apparatus can be positioned accurately at the intended target position on the body part of the user. This leads to a more consistent and reliable placement of the apparatus, which increases the efficacy of the ultrasonic treatment.

Referring to the apparatus 100 shown in FIG. 1 with the modification means 130 individually adapted to the anatomical structures of the body part of the user, the focusing of the ultrasonic waves is again illustrated by way of example in FIG. 8. The other components of the apparatus in addition to the modification means 130 are arranged in the example of FIG. 8 in a housing 810 on circuit boards. The modification means 130 is illustrated purely for reasons of better clarity in a type of exploded illustration separately from the housing 810.

The ultrasonic waves 820 are focused by the modification means 130 onto a point 825 that represents the target region. Correspondingly, the ultrasonic energy can be focused in a targeted manner onto the target region and the interaction with surrounding tissue can be reduced.

The aspects and features described in connection with a particular one of the preceding examples may also be combined with one or more of the further examples in order to replace an identical or similar feature of this further example or in order to additionally introduce the feature into the further example.

It is further understood that the disclosure of a plurality of steps, processes, operations or functions disclosed in the description or the claims is not to be interpreted as necessarily being in the described order, unless explicitly stated in the individual case or necessarily required for technical reasons. Therefore, the performance of a plurality of steps or functions is not limited to a particular order by the preceding description. Furthermore, in further examples, a single step, a single function, a single process or a single operation may include and/or be broken into a plurality of substeps, functions, processes or operations.

If some aspects have been described in the preceding portions in connection with an apparatus or a system, these aspects are also to be understood as a description of the corresponding method. In this case, for example, a block, an apparatus or a functional aspect of the apparatus or of the system may correspond to a feature, such as a method step, of the corresponding method. Accordingly, aspects described in connection with a method are also to be understood as a description of a corresponding block, a corresponding element, a property or a functional feature of a corresponding apparatus or of a corresponding system.

The following claims are hereby incorporated into the detailed description, wherein each claim may stand on its own as a separate example. Furthermore, it should be noted that—although a dependent claim in the claims relates to a specific combination with one or more other claims—other examples may also comprise a combination of the dependent claim with the subject matter of each other dependent or independent claim. Such combinations are hereby explicitly proposed, unless it is stated in the individual case that a specific combination is not intended. Furthermore, features of a claim are also intended to be included for each other independent claim, even if this claim is not directly defined as being dependent on this other independent claim.

1. An apparatus for non-invasive ultrasonic treatment of a target region within a body part of a user, comprising:
an attachment means for attaching the apparatus to the body part; and
at least one ultrasonic transducer configured to emit ultrasonic waves for non-invasive ultrasonic treatment of the target region,
wherein, for compensating for distortions of the emitted ultrasonic waves by anatomical structures of the body part and for focusing the ultrasonic waves into the target region, the apparatus:
comprises a modification means individually adapted to the anatomical structures of the body part of the user, which modification means is configured to modify acoustic properties of the emitted ultrasonic waves in a predetermined manner; or
comprises a plurality of ultrasonic transducers and a control circuit, wherein the control circuit is configured to control the emission of the ultrasonic waves by the plurality of ultrasonic transducers based on data indicating the target region and properties of the anatomical structures of the body part of the user,
wherein the apparatus is configured for operation in the magnetic field of a magnetic resonance imaging system.

2. The apparatus according to claim 1, wherein the modification means comprises at least one of an ultrasonic lens and an acoustic phase plate for predetermined modification of the acoustic properties of the emitted ultrasonic waves.

3. The apparatus according to claim 1, wherein the modification means is arranged replaceably.

4. The apparatus according to claim 1, wherein the control circuit is configured to set, based on the data, at least one of amplitude, phase, frequency and transmission pattern of the ultrasonic waves individually for the respective ultrasonic transducer.

5. The apparatus according to claim 1, wherein at least one of the plurality of ultrasonic transducers is configured to emit ultrasonic waves for determining at least one property of the anatomical structures of the body part of the user, wherein at least one of the plurality of ultrasonic transducers is configured to receive reflections of the ultrasonic waves for determining at least one property of the anatomical structures of the body part of the user.

6. The apparatus according to claim 5, further comprising a processing circuit configured to determine the data indicating the properties of the anatomical structures of the body part of the user based on the reflections measured by the at least one of the plurality of ultrasonic transducers.

7. The apparatus according to claim 5, further comprising an interface configured to output output data indicating the reflections measured by the at least one of the plurality of ultrasonic transducers to an external entity and subsequently receive the data indicating the properties of the anatomical structures of the body part of the user from the external entity.

8. The apparatus according to claim 1, further comprising an interface configured to receive the data indicating the target region from an external entity.

9. The apparatus according to claim 1, further comprising a user interface configured to receive a user input and generate the data indicating the target region based on the user input.

10. The apparatus according to claim 1, further comprising:
a contact means for contacting the body part of the user, wherein a shape of the contact means is deformable to conform to a shape of the body part of the user, and wherein the contact means is configured to acoustically couple the at least one ultrasonic transducer and the body part of the user.

11. The apparatus according to claim 1, further comprising:
an accumulator configured to provide electrical energy to the at least one ultrasonic transducer and other electrical components of the apparatus.

12. The apparatus according to claim 1, further comprising:
a housing in which the at least one ultrasonic transducer is arranged, wherein the housing extends in three mutually perpendicular spatial directions and an extension of the housing in each of the three mutually perpendicular spatial directions is less than 10 cm.

13. The apparatus according to claim 1, wherein the control circuit is further configured to control the emission of the ultrasonic waves by the plurality of ultrasonic transducers based on data indicating at least one physiological property of the user during the non-invasive ultrasonic treatment of the target region.

14. The apparatus according to claim 13, wherein the apparatus comprises at least one sensor configured to measure the at least one physiological property of the user during the non-invasive ultrasonic treatment of the target region.

15. The apparatus according to claim 13, further comprising an interface configured to receive the data indicating at least one physiological property of the user during the non-invasive ultrasonic treatment of the target region from an external entity.

16. The apparatus according to claim 1, wherein the attachment means is configured for non-invasively attaching the apparatus to the body part of the user.

17. The apparatus according to claim 1, further comprising an interface configured to exchange data for coordinated non-invasive ultrasonic treatment with another apparatus for non-invasive ultrasonic treatment.

18. The apparatus according to claim 1, further comprising:
a camera configured to capture images of an environment of the apparatus;
a processing circuit configured to:
determine a positioning of the apparatus relative to a target position on the body part of the user from the images of the camera; and
determine instructions for assisting a positioning of the apparatus at the target position based on the determined positioning of the apparatus relative to the target position; and
an output means configured to output the instructions.

19. A system comprising:
an apparatus for non-invasive ultrasonic treatment of a target region within a body part of a user, comprising:
an attachment means for attaching the apparatus to the body part; and
at least one ultrasonic transducer configured to emit ultrasonic waves for non-invasive ultrasonic treatment of the target region,
wherein, for compensating for distortions of the emitted ultrasonic waves by anatomical structures of the body part and for focusing the ultrasonic waves into the target region, the apparatus:
comprises a modification means individually adapted to the anatomical structures of the body part of the user, which modification means is configured to modify acoustic properties of the emitted ultrasonic waves in a predetermined manner; or
comprises a plurality of ultrasonic transducers and a control circuit, wherein the control circuit is configured to control the emission of the ultrasonic waves by the plurality of ultrasonic transducers based on data indicating the target region and properties of the anatomical structures of the body part of the user,
wherein the apparatus is configured for operation in the magnetic field of a magnetic resonance imaging system; and
a position tracking system configured to:
determine a position of the apparatus relative to a target position on the body part of the user; and
output information about the position of the apparatus relative to the target position.
